(19) United States
(12) Patent Application Publication
(10) Pub. No.: US 2009/0060854 A1
(43) Pub. Date: Mar. 5, 2009

(54) Dermatological Compound

(76) Inventor: Michael Southard, Arlington, TX (US)

Correspondence Address: LAW OFFICES OF JERRY A. SCHULMAN, 1S376 SUMMIT AVENUE, COURT C, OAKBROOK TERRACE, IL, 60181, US

(21) Appl. No.: 12/098,241

(22) Filed: Apr. 4, 2008

Related U.S. Application Data
(60) Provisional application No. 60/939,039, filed on May 18, 2007.
(60) Provisional application No. 60/940,195, filed on May 25, 2007.

(51) Int. Cl. A61K 31/726; A61P 17/00; A61Q 17/04

(52) U.S. Cl. 424/60; 514/54; 424/59

(57) ABSTRACT

A topically-applied composition for the treatment of the dermal and subcutaneous skin layers combines selected sulfonated glycosaminoglycans and two anti-oxidizing compounds in a pharmaceutically acceptable carrier. A constituent to treat damage due to ultraviolet A and/or B light can be added to the composition. Differing formulations are provided for daytime and evening application.

The present invention relates to dermatological preparations and, in particular, to formulations used to preserve and protect the three layers of the skin, diminish or remove fine lines and wrinkles and age spots and to slow down and/or limit the aging process.

### PRIORITY

This application is a continuation-in-part of and claims priority from U.S. patent application Ser. No. 60/939,039, filed May 18, 2007, and U.S. patent application Ser. No. 60/940,195, filed May 25, 2007, both of which are hereby incorporated herein by reference.

### BACKGROUND OF THE INVENTION

The skin may exhibit damage resulting from chronological aging and/or premature aging due to environmental/external factors such as sun exposure, smoking, air pollution or rapid weight loss. There are numerous approaches to dealing with these problems including injectable fillers, implants and topical formulations.

Injectable fillers (including Botox®) are successful to some degree, however, they are temporary and the required repeated injections are costly, painful and are not without potential short and long term complications.

Implants are more permanent but require a surgical procedure which carries its own set of risks including infection, scarring and attaining and maintaining the desired aesthetic effect.

Topical formulations have been introduced that may not attain the desired effects when used alone, in combination or in a low dose. A topical product that works only on the surface with little to no penetration of the epidermis cannot address the needs of the three layers of skin affected by the aging process. Enhanced penetrability of targeted skin layers with substances that anatomically replace or mechanically repair the damaged tissue, according to the specific needs of that layer, would provide a more thorough and natural approach to the repair process.

The three layers of the skin are the outer layer (epidermis), middle layer (dermis) and the bottom layer (subcutaneous tissue).

The normal epidermis is the barrier to the external environment. Keratinocytes move from the bottom layer of the epidermis to the top layer which develops the outer shell as a barrier. Once these cells reach the top layer, they flake off. As a person ages, the epidermal cells become thinner and less adhesive. The decreased adhesiveness negatively impacts the barrier function and allows skin moisture to be released rather than retained. This causes skin dryness and there is a corresponding decrease in epidermal cells which also divide more slowly as one ages.

The epidermal layer is easiest to access with topical treatments, however it is the underlying layers that are responsible for the visible effects of aging on the epidermis. One of the antioxidants described in more detail below as, variously, fat soluble Vitamin A, retinol, tretinoin or all-trans-retinoic acid (ATRA), can provide benefit to all layers by acting as a deep penetrant carrier for other substances and also to specifically increase the rate of cell turnover, countering the aging process of decreased epidermal cells. ATRA has also been demonstrated to increase collagen and to maintain collagen's elastic state.

The second layer, or normal dermis, contains the structural elements of the skin, the connective tissue including collagen for strength of the skin, proteins called glycosaminoglycans (GAGs) for resistance to skin deformation and elastin to provide spring or elasticity.

As a person ages the dermis thins, due to a pronounced reduction of collagen and as the elastin fibers wear out. These second layer changes affect the scaffolding/support for the skin and leads to the surface wrinkles and sagging.

The dermis layer treatment requires deep penetrating substances to repair the connective tissue by regeneration of collagen and the GAG proteins for deformation resistance and which in turn support normally functioning elastin fibers.

Maintenance of the important dermal-epidermal junction is accomplished when the cells of the epidermis receive sustaining nutrients from the blood vessels located in the dermis. A series of finger-like structures called rete pegs project up from the dermis, and similar structures project down from the epidermis. These projections increase the area of contact between the layers of skin, and help to prevent the epidermis from being sheared off. The rete ridges in this junction provide increased surface area for the blood vessels to deliver the required nutrients. ATRA and alpha-tocopherol can help maintain this junction by their capability to penetrate these layers and deliver the substances described herein.

Normal subcutaneous tissue, the bottom layer of skin, contains fat cells which provide insulation and make the skin appear tight, or plump, and unwrinkled. With aging, the fat cells in the subcutaneous layer get smaller. This leads to wrinkles and sagging because the fat cells can no longer fill in or ‘correct’ the damage to the epidermal or dermis layers.

A preferred formulation for treatment of the three skin layers with a single application comprises two or more members of the glycosaminoglycan family, either of which may be sulfated. This aspect of the formulation is designed to contribute to cell proliferation and migration and is an important component of skin and connective tissues.

Also included in the formulation are two or more antioxidants which counteract the overproduction of free radicals (oxidants), which in turn, activate negative metalloproteinases that degrade connective tissue and contribute to wrinkling. The antioxidants are selected to produce both daytime and nighttime formulations.

Substances are also included in the formulation to block ultraviolet radiation in the A and B bands.

Substances are also selected for positive moisturizing effect, whitening effect, UV absorbing effect, anti-oxidant effect, hair growing effect, wrinkle reducing effect, cell activating effect and site-specific transdermal absorption effect.

Tretinoin, also known as all-trans-retinoic acid (ATRA) is commonly known as a medication used to treat acne vulgaris and keratosis pilaris. It is sold under the brand name Retin-A. ATRA is known to penetrate deep beneath the skin to treat wrinkles, act as an anti-oxidant to reduce the presence of free radicals and has also been demonstrated to provide a collagen boost.

Rooibos (aspalathus linearis) is a member of the legume family of plants, most often used to make an herbal tea preparation, commonly called African Red Tea. Rooibos contains a high level of anti-oxidants such as superoxide dismutase. Rooibos exhibits many of the anti-oxidant properties of ATRA.

A second substance, alpha-tocopherol (fat soluble Vitamin E), provides additional powerful antioxidant support to assist cell turnover and normalization of connective tissue.

The present invention has as an objective to provide an external, topically-applied dermatological formulation to treat all three skin layers at the same time. The formulation preferably has enhanced dermal penetrability and ingredients that have proven positive effects on the individual requirements of each of the three layers of skin that are damaged during the aging process. The combination provides replacement latticework and structure to the collagen foundation as well as the elastin fibers and the fat cells. The antioxidants activate metalloproteinases that provide positive remodeling of the collagen structure and enhance penetration as a carrier and site specific delivery vehicle to the targeted skin layer where specific repair functions are required by each respective layer.

The GAGs contained in the formulation are targeted to specific requirements of the dermis layer and subsequently the bottom subcutaneous layer. They also have considerable water holding capacity to ensure that the skin retains its normal water-lipid balance.

GAGs form an important component in connective tissues. Specific to the dermal layer, the single most helpful GAG is chondroitin sulfate which provides resistance to compression and provides the structural integrity required for that specific anatomical space as well as the mechanical support for normal skin processes to take place. This contributes to ensuring healthy functioning sebaceous glands, sweat glands and hair shafts. Interrupting the degradation process with a replica process that can be administered and maintained with a topical formulation is essential to controlling the chronological and premature aging process.

Chondroitin sulfate is a sulfated GAG composed of a chain of alternating sugars and is a naturally-occurring substance in the body. It is an important structural component of cartilage and provides cartilage with much of its resistance to compression Chondroitin sulfate is well represented in the prior art as a dermatologically useful substance.

Problems in the sub-cutaneous layer are addressed with another GAG substance known as hyaluronan, or hyaluronic acid. Hyaluronan is a non-sulfated GAG and is found in the body in connective, epithelial and neural tissues. Hyaluronan is also found in the body's synovial fluid and it has been demonstrated that hyaluronan increases the viscosity of synovial fluid and, along with lubricin, is one of the synovial fluids main lubricating components.

Hyaluronan acts as a coating for articular cartilage. Hyaluronan is also a major component of skin, where it is involved in tissue repair. When skin is excessively exposed to ultraviolet B rays, sunburn results and the cells in the skin stop producing as much hyaluronan and increase the rate of its degradation. Hyaluronan degradation products also accumulate in the skin after UV exposure.

Hyaluronan has a well recognized role as a biomaterial scaffold for tissue. Hyaluronan also contributes to tissue hydrodynamics and cell proliferation, therefore it is well suited to provide the volume required to support and replace, as required, the shrinking of fat cells seen in the aging process.

In addition to these GAG ingredients being optimally absorbed through the ATRA, rooibos and alpha-tocopherol carrier function, there is epidermal benefit from the moisturizing effect of this topical formulation.

In summary, each layer of the skin must be treated specifically to maintain the structural integrity of the overall tissue and to contribute to the normal skin regeneration process. Structural integrity and functional replacement, within each layer, with highly absorbed topical substances, can successfully promote a healthy skin process.

An additional feature of the foregoing formulation is the addition of substances to furnish additional protection from the effects of medium wave ultraviolet radiation (UVB) on the skin. As described in the Wikipedia topic heading for ultraviolet light:
“When considering the effect of UV radiation on human health and the environment, the range of UV wavelengths is often subdivided into UVA (400-320 nm), also called Long Wave or black light; UVB (320-280 nm), also called Medium Wave; and UVC (<280 nm), also called Short Wave or “germicidal”. See 1 E-7 m for a list of objects of comparable sizes.
“UVA, UVB and UVC can all damage collagen fibers and thereby accelerate aging of the skin. In general, UVA is the least harmful, but can contribute to the aging of skin, DNA damage and possibly skin cancer. It penetrates deeply and does not cause sunburn. Because it does not cause reddening of the skin (erythema) it cannot be measured in the SPF testing. There is no good clinical measurement of the blocking of UVA radiation, but it is important that sunscreen block both UVA and UVB.
“UVA light is also known as “black light” and, because of its longer wavelength, can penetrate many windows. It also penetrates deeper into the skin than UVB light and is thought to be a prime cause of wrinkles.
“The reddening of the skin due to the action of sunlight depends both on the amount of sunlight as well as the sensitivity of the skin (“erythemal action spectrum”) over the UV spectrum.”

I have found that combining the foregoing substances in a dermatological preparation results in significant benefits. For example preparations formulated with the foregoing ingredients can be used topically, eliminating the need for injecting a dermatological product when seeking to control or eliminate wrinkles. As used topically, the formulations can be supplied as creams, lotions, wipe-on pads, overnight treatment strips, gels, masks and the like.

The formulation is provided in daytime and nighttime variations: the daytime formula eliminates ATRA due to its known photosensitivity (susceptibility to sunburn) and replaces it with rooibos, another powerful antioxidant that is capable of promoting absorption . The daytime formula would also contain a sunscreen.

Descriptions of the foregoing compounds and the conditions they are intended to treat are well-represented in the following prior art references, all of which are incorporated herein by reference:

United States patent application publication 2007/0003502 (Tanabe, et al.) teaches and describes a skin preparation for external use characterized by containing sugar derivative of alpha, alpha-trehalose.

United States patent application publication 2006/0165636 teaches and describes hair treatment compositions and hair cosmetic for damaged hair.

United States patent application publication 2005/0118283 (Calverly, et al.) teaches and describes skincare compositions and methods.

United States patent application publication 2005/0118283 (Calverly) teaches and describes skincare compositions and methods.

United States patent application publication 2007/0292527 (Christensen) teaches and describes composition for the cosmetic treatment of age-related dermatological symptoms.

U.S. Pat. No. 6,984,390 (Sakuta) teaches and describes a silicone compound and cosmetic preparation.

United States patent application publication 2003/0095940 (Schiltz), U.S. Pat. No. 7,175,837 (Schiltz) and U.S. Pat. No. 6,495,126 all teach and describes teaches and describes a treatment and composition for achieving skin anti-aging benefits by corneum protease activation.

U.S. Pat. No. 6,541,614 (Nagasawa, et al.) teaches and describes a polysacchride derivative for use in toiletries.

U.S. Pat. No. 6,782,307 (Wilmott) teaches and describes a method for producing customized and pharmaceutical formulations on demand.

United States patent application publication 2005/0003030 (Simon, et al.) teaches and describes antioxidant and anti-inflammatory activity of compounds and preparations from African nutmeg seeds.

### DETAILED DESCRIPTION OF AN EMBODIMENT OF THE INVENTION

A preferred nighttime preparation includes chondroitin sulfate at about 4.0 percent by weight, hyaluronan at about 2.5 to 3.0 percent by weight, ATRA at about 0.05 percent by weight, and alpha-tocopherol at about 5.0 percent by weight.

Because ATRA is known to increase photosensitivity, a daytime version substitutes rooibos for ATRA. A suitable sunscreen can also be included in the daytime formula, typically in an amount of about 3.0 percent by weight.

A preferred daytime preparation includes chondroitin sulfate at about 4.0 percent by weight, hyaluronan at about 2.5 to 3.0 percent by weight, rooibos at about 0.05 percent by weight, and alpha-tocopherol at about 5.0 percent by weight.

The remaining constituents of a selected preparation includes a carrier consisting of water water (about 22.279 percent by weight), propylene glycol (about 2.000 percent by weight), methylparaben (about 0.450 percent by weight), sodium borate (about 0.500 percent by weight), aerosyl 200 (about 1.500 percent by weight), dicapryl maleate (about 8.000 percent by weight), hydrogenated polyisobutane (about 13.600 percent by weight), vegetable oil (about 7.000 percent by weight), acetylated lanolin (about 4.000 percent by weight), propylparaben (about 0.038 percent by weight), ethylparaben (about 0.113 percent by weight), BHT (about 0.020 percent by weight), cholesterol (about 0.250 percent by weight), Elfacos® ST 37 (about 2.000 percent by weight), amphisol (about 0.300 percent by weight), petrolatum (about 7.900 percent by weight), beeswax (about 6.000 percent by weight),and Miglyol® gel (about 8.500 percent by weight). These percentages may vary slightly depending upon the proportions of the actual ingredients discussed above.

The formulation also includes an appropriate fragrance in an amount of about 0.500 percent by weight and may also include Germall® 115 (about 0.300 percent by weight).

Thus, combining chondroitin sulfate, hyaluronan, ATRA or rooibos, together with a second anti-oxidant such as alpha-tocopherol in a carrier generally as described above, provides a topical anti-wrinkle formulation which may be used without the need for injections.

Variations on the foregoing formulations includes a UV-specific chromophore whose function would be to absorb or block UVA and/or UVB radiation to act as a preventive for skin aging. The chromophore “filters” a percentage of the UV spectrum depending on the type and intensity of chromophores used. The usable chromophore family includes (alone or in combination) the benzotriazoles, benzophenones, benzoic acids/PABA, cinnamates and salicylates and avobenzones. The chromophore acts as an additive that would further protect against UVA and UVB damage without being an opaque block.

Preferably, the UVA blocker would be present in an amount of about 3.0 percent by weight When both a UVA and UVB blocker are used each would be present in an amount of about 1.5 percent by weight.

I claim:

1. A composition comprising:
chondroitin sulfate in a pharmaceutically effective amount to treat the dermal skin layer;
hyaluronan in a pharmaceutically effective amount to treat the subcutaneous skin layer;
a first anti-oxidant compound;
at least a second anti-oxidant compound; and
a pharmaceutically acceptable carrier for applying said composition topically.

2. The composition of claim 1 further comprising at least one constituent to block ultraviolet light.

3. The composition of claim 2 wherein said UV blocking constituent is present in amount of about 3.0 percent by weight.

4. The composition of claim 2 further comprising a first UV blocker for blocking UVA radiation and a second UV blocker for blocking UVB radiation.

5. The composition of claim 4 wherein said UVA and UVB blockers are each present in an amount of about 1.5 percent by weight.

6. The composition of claim 2 wherein said UV blocking constituent is a UV specific chromophore.

7. The composition of claim 6 wherein said chromophore includes at least one of the following: the benzotriazoles, the benzophenones, benzoic acid, PABA, the cinnamates, the salicylates or the avobenzones.

8. A composition comprising:
chondroitin sulfate in an amount equal to about 4.0 percent by weight;
hyaluronan in an amount equal to about 2.5 to 3.0 percent by weight;
all-trans-retinoic acid in an amount equal to about 0.05 percent by weight;
alpha tocopherol in an amount equal to about 5.0 percent by weight; and
a pharmaceutically acceptable carrier for applying said composition topically.

9. The composition of claim 8 further comprising a constituent to block ultraviolet light.

10. The composition of claim 8 wherein said UV blocking constituent is present in amount of about 3.0 percent by weight.

11. The composition of claim 8 further comprising a first UV blocker for blocking UVA radiation and a second UV blocker for blocking UVB radiation.

12. The composition of claim 11 wherein said UVA and UVB blockers are each present in an amount of about 1.5 percent by weight.

13. The composition of claim 9 wherein said UV blocking constituent is a UVA specific chromophore.

14. The composition of claim 13 wherein said UVA specific chromophore includes at least one of the following: the benzotriazoles, the benzophenones, benzoic acid, PABA, the cinnamates, the salicylates or the avobenzones.

15. A composition comprising:
chondroitin sulfate in an amount equal to about 4.0 percent by weight;
hyaluronan in an amount equal to about 2.5 to 3.0 percent by weight;
rooibos in an amount equal to about 0.05 percent by weight;
alpha tocopherol in an amount equal to about 5.0 percent by weight; and
a pharmaceutically acceptable carrier for applying said composition topically.

16. The composition of claim 15 further comprising a constituent to block ultraviolet light.

17. The composition of claim 16 wherein said UV blocking constituent is present in amount of about 3.0 percent by weight.

18. The composition of claim 16 further comprising a first UV blocker for blocking UVA radiation and a second UV blocker for blocking UVB radiation.

19. The composition of claim 18 wherein said UVA and UVB blockers are each present in an amount of about 1.5 percent by weight.

20. The composition of claim 16 wherein said UV blocking constituent is a UVA specific chromophore.

21. The composition of claim 20 wherein said chromophore includes at least one of the following: the benzotriazoles, the benzophenones, benzoic acid, PABA, the cinnamates, the salicylates or the avobenzones.
